(19) United States
(12) Patent Application Publication
(10) Pub. No.: US 2024/0002470 A1
(43) Pub. Date: Jan. 4, 2024

(54) Method for separation and/or detection and/or in vitro quantification of infectious compounds in biological material

(71) Applicant: APOH-TECHNOLOGIES, LA GRANDE-MOTTE (FR)

(72) Inventor: Elias STEFAS, LA GRANDE-MOTTE (FR)

(21) Appl. No.: 18/040,945

(22) PCT Filed: Aug. 5, 2021
(86) PCT No.: PCT/EP2021/071917

§ 371 Date: Feb. 7, 2023

Related U.S. Application Data
(60) Provisional application No. 63/064,244, filed on Aug. 11, 2020.

(51) Int. Cl. C07K 14/775; G01N 33/569

(52) U.S. Cl. CPC C07K 14/775; G01N 33/569; G01N 2333/775

(57) ABSTRACT

The present invention relates to apolipoprotein H-based peptides capable of binding microorganisms, a method of separation and/or detection and/or quantification and/or in vitro identification of infectious compounds in a biological material using the same. Particularly the invention relates to peptides capable to bind microorganisms and their use as sensors to capture microorganisms present in a biological material.

The present invention relates to a method of separation and/or detection and/or quantification and/or in vitro identification of infectious compounds in a biological material.

In the present patent application, the term “biological material” means a biological tissue, a preparation or an extract derived from biological tissue, liquid or solid, or a medium, natural or not, capable of containing infectious compounds, for example a runoff or fruit and vegetable rinse water. Such a material can also be a mixture of at least two materials as defined above; it can therefore be, in particular, either prepared from tissues, organs, stool or body fluids of a patient suffering from an infection, or obtained from “in vitro” cultures; such a biological material can also be a serum, plasma, urine, cerebrospinal fluid, synovial fluid, peritoneal fluid, pleural fluid, seminal fluid or ascetic fluid.

In the present patent application, the term “infectious compounds” means infectious agents, exogenous or endogenous, or their metabolites; among the infectious compounds that may be mentioned, for example, viruses, bacteria or fungi.

It has been already described a plasma glycoprotein called β2-glycoprotein I, or abbreviated as “β2GPI”; the sequence of this human glycoprotein has in particular been indicated in the articles by J. LOZIER et al., Proc. Natl. Acad. Sci. USA, Vol. 81, p. 3640-3644 (1984) and T. KRISTENSEN et al., FEBS Letters, Vol. 289, p. 183-186 (1991). It was found that this β2GPI protein has a polymorphism: the name β2GPI will be considered below as generic for all forms.

In international application WO 94/18569, it was indicated that certain infectious compounds, in particular proteinaceous compounds, were fixed on the form of β2GPI which had been described in French patent 2 701 263. It was proposed in document WO 94/18569, a method for detecting and/or assaying viral compounds in which the infectious viral compounds are fixed on the form of β2GPI used; this form of β2GPI is therefore added to viral infectious compounds contained in a biological material, so as to separate the viral compounds thus captured so as to then detect and/or measure them. In European patent EP 0775 315, the formation of a complex has been described between an infectious compound, in particular a proteinaceous compound, and any form of β2GPI; the infectious compound could, in particular, be a bacterium or a virus. At the present time, the role of β2GPI in vivo is poorly understood. However, in vitro studies have shown that β2GPI has the particularity of binding to negatively charged structures and molecules, in particular anionic phospholipids (PL), platelets, apoptotic cells, mitochondria, DNA, biliary acids . . . . In certain autoimmune pathologies, such as antiphospholipid syndrome (SAP), antibodies, directed against β2GPI or against the β2GPI-PL complex, have been highlighted. These antibodies lead to an inhibition of coagulation, hence their name of circulating anticoagulants. They are associated with thrombotic, venous or arterial clinical manifestations and recurrent fetal loss. SAP can be accompanied by various clinical manifestations: thrombocytopenia, coronary or valvular disorders, neurological disorders, autoimmune hemolytic anemias . . . . The presence of these antibodies can also be encountered during infectious diseases (viral, bacterial or parasitic) and neoplasias (solid tumors, lymphoproliferative syndromes, etc.). These properties are summarized in the article by S. Miyakis et al., Thrombosis Research, Vol. 114, Issues 5-6, 2004, p. 335-346.

In order to determine the β2GPI epitopes involved in SAP several studies have been carried out. Thus Gharavi et al., Journal of Autoimmunity (2000) 15, p. 227-230, by immunizing mice with the peptide of 15 amino acids (aa) between Gly274 and Cys288, from the Vth domain of β2GPI, induce anti-PL antibodies and anti-β2GPI antibodies. This region corresponds to that described by Hunt and Krilis, Journal of Immunology, 1994 Jan. 15; 152 (2): 653-9, and who is responsible for liaison with the PL. Ito et al., Human Immunology (2000) 61 (4), p. 366-77, using peptide libraries, identified the sequences of β2GPI, involved in the response of T cells. Blank et al., PNAS (1999) 96 (9), p. 5164-5168, identified 3 peptide sequences in β2GPI which would be involved in the SAP. Pope et al., J. Immunol. (2012) 189, p. 5047-5168, describe the use of peptides from the Vth domain of β2GPI to reduce inflammation in the context of intestinal ischemia. Finally, Du et al., Br J Haematol. (2012) 159 (1), p. 94-103, show that domain I of β2GPI would have a protective effect in thrombocytopenic thrombotic purpura. Nilsson et al., Mol Microbiol. (2008) 67 (3), p. 482-92, demonstrate the bactericidal effect of certain peptides derived from the V domain of β2GPI. Assuming that antibacterial peptides have a common structure with groups of cationic and hydrophobic amino acids, Yeaman and Yount, Pharmacol Rev. (2003) 55 (1), p. 27-55, study 6 peptides: SRGGMRK (SRG7) present in domain I of β2GPI and GDKVSFFCKNKEKKCS (GDK15), GDKVSFF (GDK7), CKNKEKKCS (CKN8), FKEHSSLAFWKTDASDVKPC (FKE20) and FKEHSSLAFWK (FKE11) present in domain V of the β2GPI. Among these peptides, only GDK15 and CNK8 have a bactericidal effect on E. coli and S. pyogenes. This effect is less for S. aureus. The other peptides have little or no effect. The percentage of bacterial survival after contact with the GDK15 peptide is different depending on the strain (for the three bacterial strains tested). All these results assume that the peptide-bacteria interaction is not the same for the 3 strains tested. Agar et al., Blood (2011) 117 (25), p. 6939-47, describe the binding of LPS of Gram negative bacteria, to the V domain of β2GPI and more precisely they describe a peptide, LAFWKTDA, of this domain capable of inhibiting the binding of β2GPI to LPS: Agar and coll., Thromb Haemost. (2011) 106 (6), p. 1069-75. It appears from these documents that 2 domains of β2GPI, domains I and V, as well as peptides located in these domains, are involved in different protein mechanisms: binding to PL, induction of anti-PL, response of T cells, bactericidal effect, binding to LPS, etc. In the study described by Nilsson, peptides have a bactericidal effect, this effect depends on the dose and the bacterial strain studied. Agar in its study highlights the binding of β2GPI, to the LPS compound found only in Gram negative bacteria. However, none of these studies formally demonstrates the binding of these peptides to bacteria as well as their use for the diagnosis of these same microorganisms. In Nilsson's study it was shown that β2GPI binds to the H and M1 proteins of S. pyogenes, but the peptide region of β2GPI responsible for this binding has not been studied. Zhang et al., Microbiology (1999) 145 (1), p. 177-83, has shown the binding of β2GPI with the Sbi protein of S. aureus. From these studies, the linkage of β2GPI with different bacterial proteins has emerged. These proteins have a priori nothing in common, therefore for a person skilled in the art several sequences should be involved in this binding. Furthermore, if the bactericidal effect of β2GPI is real, the bacteria-peptide bond (corresponding to the V domain of β2GPI) would not allow the detection by culture, of bacteria captured by supports on which these peptides are grafted. Consequently, taking into account the facts described above, a person skilled in the art would not have been inclined to seek a fixation of bacteria on β2GPI peptides, common for all the bacteria, for the purposes of separation and/or for detecting and/or identifying and/or quantifying bacteria, in a biological material. Finally, there have been no studies concerning the binding of peptides corresponding to regions of β2GPI to various viruses and fungi. As these are completely different microorganisms, viruses, bacteria and fungi, a person skilled in the art would not have been inclined to seek a fixation of bacteria, viruses or fungi on β2GPI peptides, common for all bacteria, viruses and fungi for the purpose of separation and/or detection and/or identification and/or quantification of bacteria, in a biological material. According to the present invention the applicant has shown that peptides, corresponding to the V domain of β2GPI and hereinafter designated by the generic term pepβ2GPI, eventually coupled to solid supports, have the property of binding bacteria, Gram+ and Gram−, as well as DNA viruses and RNA viruses and fungi.

According to the present invention, this property is used to capture bacteria, viruses and fungi, detect and/or quantify them by molecular biology techniques (PCR, RT-PCR), immunoenzymatic, by measurement of ATP, by culture . . . .

Thus the present invention relates to peptides capable of binding microorganisms chosen from
P1: SSLAFWK
P2: CKNKEKKC in cyclic form (presence of a disulfide bridge)
P3: CKNKEKKC in linear form (absence of disulfide bridge) alone or linked to each other to give the following molecules
P4: CKNKEKKCGGSSLAFWK; or
P5: R—X—R′
P6: H—(R)₂—K or
P7: H—[(R)₂—K)₂—K
Wherein
R and R′, identical or different, can be P1: SSLAFWK; or P2: CKNKEKKC in cyclic form; or P3: CKNKEKKC in linear form; and
X can be one spacer chosen from
   Sp1: —CO—NH—(CH₂)m-NH—CO—(CH₂)n-CO—NH—(CH₂)p-CO—NH—; wherein m can be an equal integer which can have any one of the values 1, 2, 3, 4, 5, 6, 7, 8, 9 or 10, preferably 4, 5, 6, 7 or 8, more preferably 6; n can be an equal integer which can have any one of the values 1, 2, 3, 4 or 5, preferably 2, 3 or 4, more preferably 3; and p can be an equal integer which can have any one of the values 1, 2, 3, 4, 5, 6, 7, 8, 9 or 10, preferably 4, 5, 6, 7 or 8, more preferably 5;
   Sp2: —CO—(CH₂)m-NH—CO—(CH₂)n-CO—NH—(CH₂)p-NH—; wherein m and p can be an equal integer which can have independently any one of the values 1, 2, 3, 4, 6, 7, 8, 9 or 10, preferably 4, 5, 6, 7 or 8, more preferably 5; and n can be an equal integer which can have any one of the values 1, 2, 3, 4 or 5, preferably 2, 3 or 4, more preferably 3;
   Sp3: —CO—(CH₂)m-O—(CH₂)n-NH—CO—(CH₂)p-O—(CH₂)q-CO—NH—(CH₂)r-O—(CH₂)s-NH—; wherein m, and q can be an equal integer which can have independently any one of the values 1, 2 or 3, preferably 2; and p and q can be an equal integer which can have independently any one of the values 1 or 2, preferably 1;
   Sp4: —CO—(CH₂)m-NH—CO—(CH₂)n-CZ—(CH₂)p-NH—CO—(CH₂)q-NH—, wherein Z=cyclic C₅H₁₀ and wherein m and q can be an equal integer which can have independently any one of the values 1, 2, 3, 4, 5, 6, 7, 8, 9 or 10, preferably 4, 5, 6, 7 or 8, more preferably 5; and n and p can be an equal integer which can have independently any one of the values 1 or 2, preferably 1;
   Sp5: —CO—(CH₂)m-NH—CO—NH—(CH₂)n-CO—NH—(CH₂)p-NH—; wherein m and p can be an equal integer which can have independently any one of the values 1, 2, 3, 4, 5, 6, 7, 8, 9 or 10, preferably 4, 5, 6, 7 or 8, more preferably 6; and n can be an equal integer which can have any one of the values 2, 3, 4, 5 or 6, preferably 3, 4, 5, more preferably 4;
   Sp6: —CO—(CH₂)m-NH—CO—(CH₂)n-NH—CO—(CH₂)p-NH—; wherein m, n and p can be an equal integer which can have independently any one of the values 1, 2, 3, 4, 5, 6, 7, 8, 9 or 10, preferably 3, 4, 5, 6 or 7, more preferably 5;
   Sp7: —CO—(CH₂)m-NH—CO—(CH₂)n-NH—; wherein m and n can be an equal integer which can have independently any one of the values 1, 2, 3, 4, 5, 6, 7, 8, 9 or 10, preferably 3, 4, 5, 6 or 7, more preferably 5;
   Sp8: —CO—(CH₂)m-CO—NH—(CH₂)n-NH—CO—(CH₂)p-NH—; wherein m can be an equal integer which can have any one of the values 1, 2, 3, 4, 5, 6, 7, 8, 9 or 10, preferably 4, 5, 6, 7 or 8, more preferably 5; n can be an equal integer which can have any one of the values 1, 2, 3, 4 or 5, preferably 2, 3 or 4, more preferably 3; and p can be an equal integer which can have any one of the values 1, 2, 3, 4, 5, 6, 7, 8, 9 or 10, preferably 4, 5, 6, 7 or 8, more preferably 6;
   Sp9: —CO—(CH₂)m-NH—CO—(CH₂)n-NH—CO—(CH₂)p-NH—; wherein m can be an equal integer which can have any one of the values 1, 2, 3, 4, 5, 6, 7, 8, 9 or 10, preferably 4, 5, 6, 7 or 8, more preferably 5; n can be an equal integer which can have any one of the values 1, 2, 3, 4 or 5, preferably 2, 3 or 4, more preferably 3; and p can be an equal integer which can have any one of the values 1, 2, 3, 4, 5, 6, 7, 8, 9 or 10, preferably 4, 5, 6, 7 or 8, more preferably 6;
   Sp10: —CO—(CH₂)m-O—(CH₂)n-O—(CH₂)p-O—(CH₂)q-O—(CH₂)r-O—(CH₂)s-NH—; wherein m, n, p, q, r, and s can be an equal integer which can have independently any one of the values 1, 2 or 3, preferably 2;
   SP11: —CO—(CH₂)m-NH—CO—(CH₂)n-NH—CO—(CH₂)p-NH—CO—(CH₂)q-NH—CO—(CH₂)r-NH—CO—(CH₂)s-NH—; wherein m, n, p, q, r, and s can be an equal integer which can have independently any one of the values 1 or 2, preferably 1;
   SP12: —CO—CH₂—NH—CO—CHCH₂OH—NH—CO—CH₂—NH—CO—CHCH₂OH—NH—CO—CH₂—NH—CO—CHCH₂OH—NH—;
   SP13: —CO—(CH₂)m-NH—CO—C[NH—CO—(CH₂)n-NH—](CH₂)p-NH—CO—(CH₂)q-NH—; wherein m, n, p and q, can be an equal integer which can have independently any one of the values 1, 2, 3, 4, 5, 6, 7, 8, 9 or 10, preferably 4, 5, 6, 7 or 8, more preferably 5;
it being understood that in P1, P2, P3, any amino acid, but only one each time, can be substituted by any other amino acid.

Preferably, according to the present invention, the peptides can be
P1: SSLAFWK
P2: CKNKEKKC in cyclic form (presence of a disulfide bridge)
P3: CKNKEKKC in linear form (absence of disulfide bridge)
P4: CKNKEKKCGGSSLAFWK;

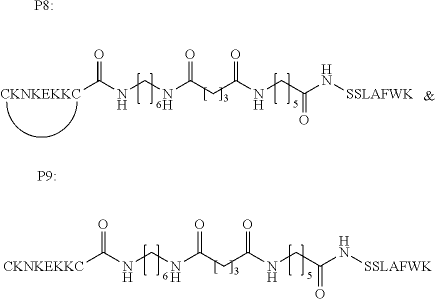

P10: H-(SSLAFWK)2-K—NH₂
P11: H-((SSLAFWK)2-K)2-K—NH₂; or
peptides comprising A: CKNKEKKC and B: SSLAFWK separated by different spacers:

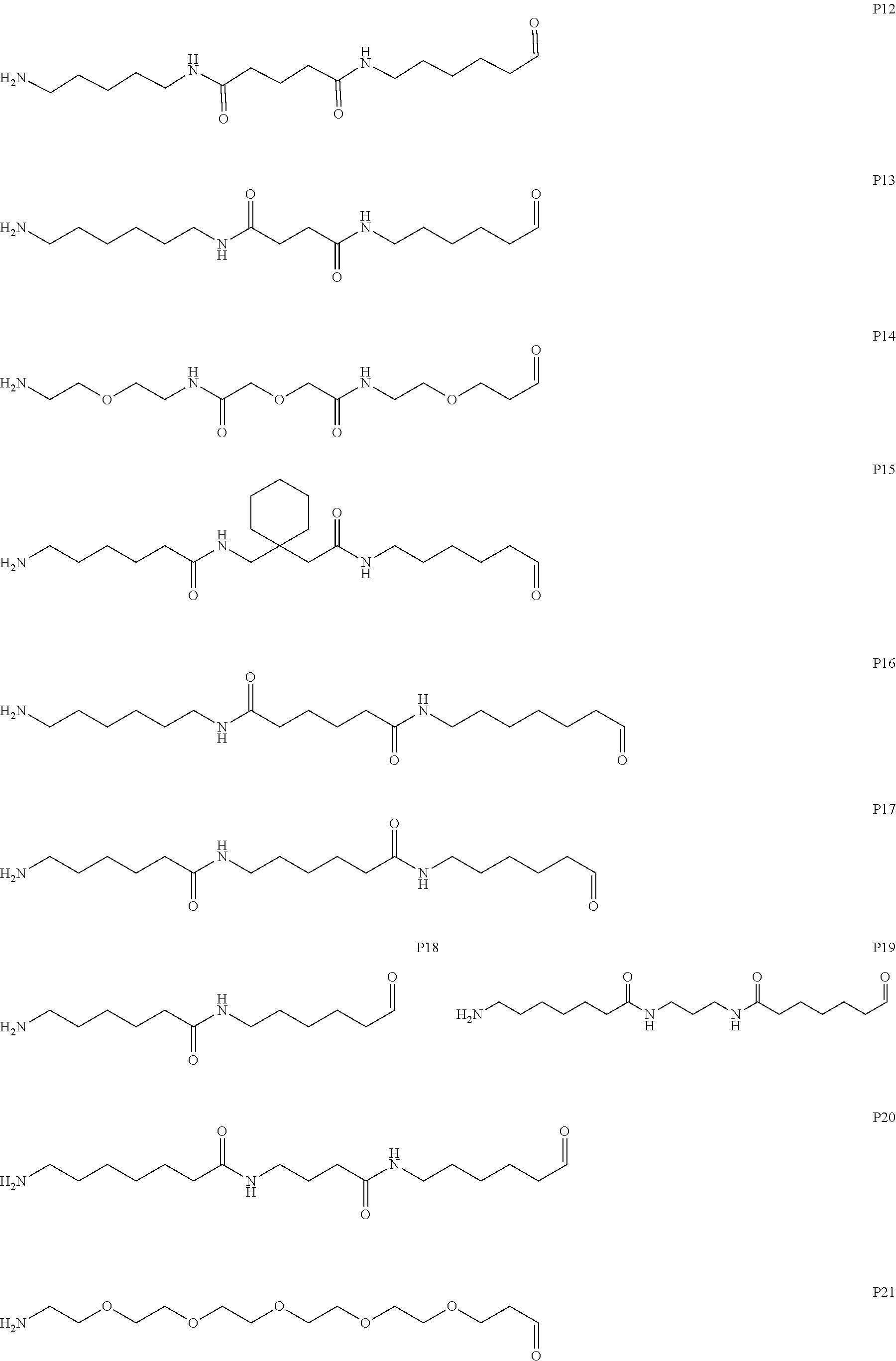

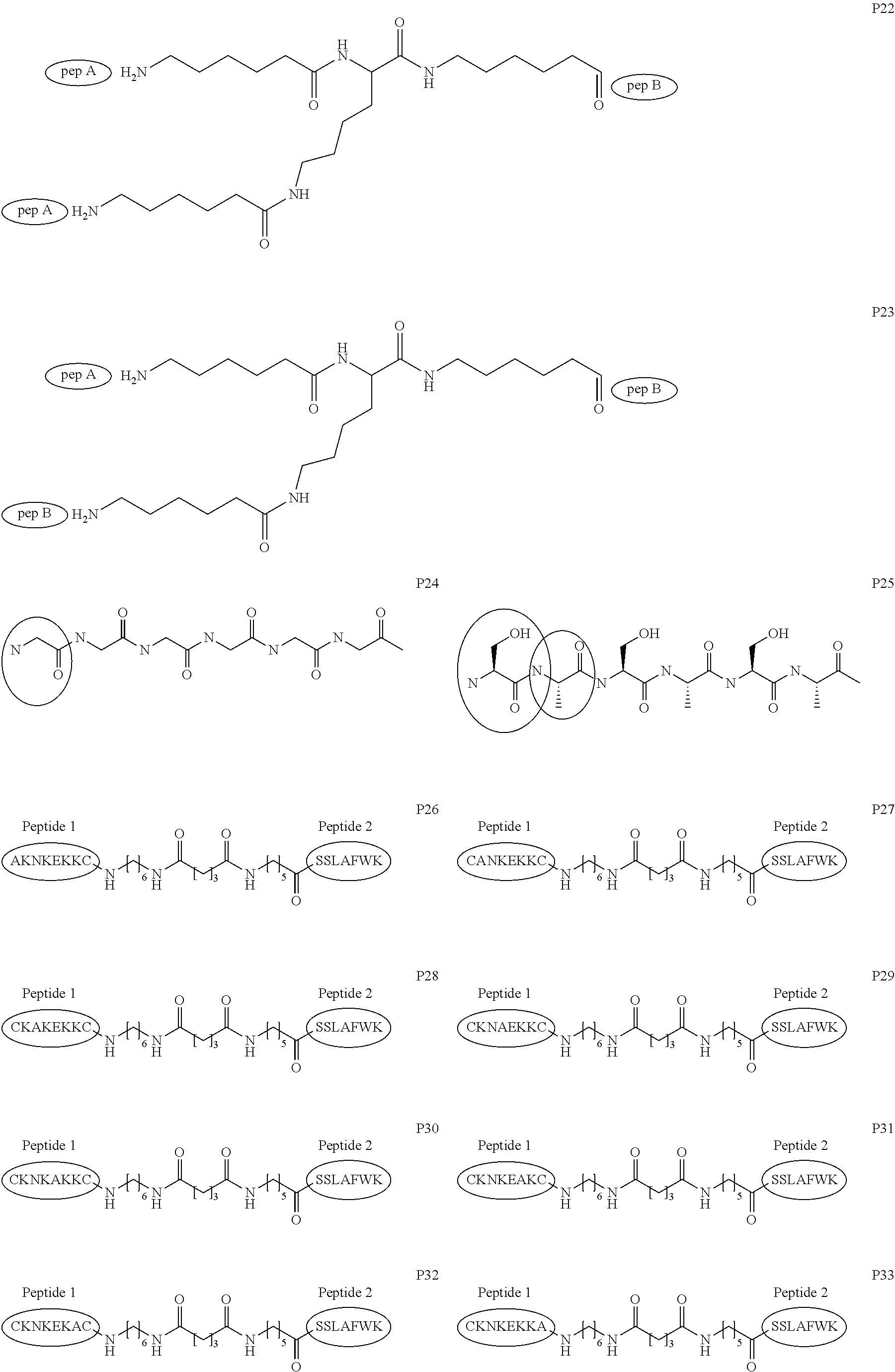

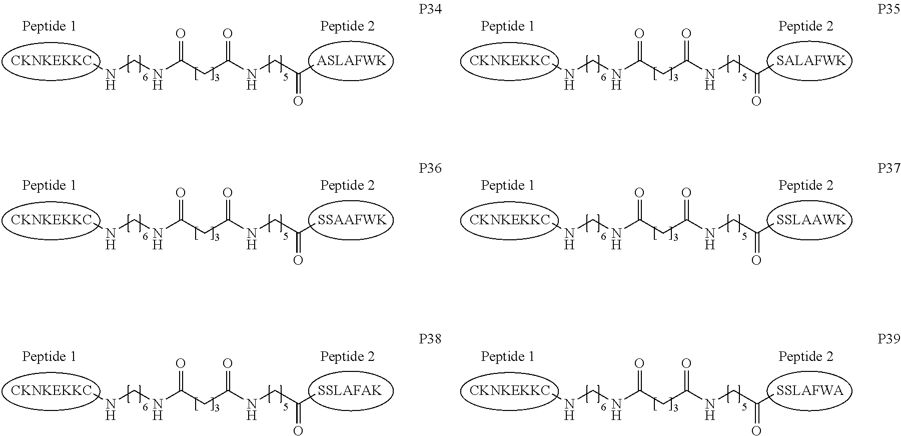

The invention also relates to peptides as describes above for their use to capture a microorganism present in biological material. According to the invention said microorganism can be a bacterium, Gram+ and Gram−, a DNA or RNA virus or a fungus. The invention also relates to a microorganism sensor, characterized in that it comprises at least one peptide as previously described coupled to a solid support.

The present invention relates to a method of separation and/or detection and/or identification and/or quantification of bacteria, viruses and fungi, in a biological material comprising peptides, pepβ2GPI, as described above, and a support solid for the implementation of the process.

Said method is preferably performed in vitro.

The term “solid support” refers to any solid support known in the field such as one of those described in “Current Protocols in Immunology by Editions Coligan J., Bierer B., Margulies D., Shevach E., Strober W., and Coico R., Wiley Interscience, 2013”. This support can for example be an ELISA type microtiter plate, a membrane, for example nitrocellulose, a chromatography gel, beads, for example made of polystyrene, tubes, for example made of polystyrene or polypropylene . . . .

According to the invention said in vitro method for capturing a microorganism present in a biological material comprises
A first step of bringing a microorganism sensor according to claim 3 into contact with biological material potentially containing microorganisms;
A second stage of incubation of the sensor and the biological material for a period of time, between 2 minutes and 24 hours;
A third step of separation of the biological material and the sensor;
A fourth step of detection and/or identification and/or quantification of the microorganisms attached to the sensor.

The attachment to the solid support of one or more of the peptides, pepβ2GPI, as described above, is carried out by reaction of reactive groups of the peptide (s), pepβ2GPI, with reactive sites of the support according to any method known in the field such as those described in “Current Protocols in Immunology by Editions Coligan J., Bierer B., Margulies D., Shevach E., Strober W., and Coico R., Wiley Interscience, 2013”. This reaction is preferably carried out at a temperature between 0° C. and 40° C., the peptide (s), pepβ2GPI, being preferably placed in a buffer having a pH between 2.5 and 10.5, preferably between 5.5 and 7.5. Preferably, an isotonic or almost isotonic buffer is used. The buffer can be of the phosphate or acetate type. The solution obtained advantageously has a concentration of between 0.005 and 100 g/l of peptides, pepβ2GPI. The support is advantageously kept in contact with the buffer containing the peptide (s), pepβ2GPI, at a temperature between 0 and 40° C. and during an incubation time between 30 minutes and 24 hours. After incubation, the buffer containing the peptide (s), pepβ2GPI, which has not reacted, is separated from the support and the support is washed, preferably with the same buffer as that which contained the peptide (s). It may be necessary to saturate the active sites of the support which have not reacted with the peptide (s), pepβ2GPI. In this case, other active groups chosen from solutions of bovine albumin, fetal calf serum, casein, glycine, detergents such as Tween 20 or 80, Triton X100 are reacted on these active sites . . . .

A solution of bovine serum albumin is advantageously used for this purpose, in particular a 2% solution in the buffer used for the peptide (s), pepβ2GPI. After reaction, the support is also preferably rinsed and dried.

The solid support reaction carrying one or more peptides, pepβ2GPI, as described above, with the biological material is carried out according to any process known in the field such as those described in “Current Protocols in Immunology by Editions Coligan J., Bierer B., Margulies D., Shevach E., Strober W., and Coico R., Wiley Interscience, 2013”. The support, on which the peptide (s), pepβ2GPI, is fixed, is then brought into contact with a biological material capable of containing bacteria. The biological material is preferably diluted using a buffer giving a pH between 3.5 and 10, advantageously between 5.6 and 7.6. The reaction is preferably carried out at a temperature between 0° C. and 50° C., advantageously close to 37° C., for a period of time, between 2 minutes and 24 hours. The biological material is then separated from the support carrying the peptide or peptides, pepβ2GPI, which has or have optionally fixed at least one bacterium. It is then optionally washed with a solution, preferably buffered.

The separation or isolation of the infectious compound (s) fixed on the solid support containing the peptide (s), pepβ2GPI, can be done according to any elution method used for affinity chromatography such as those described in “Guide to protein purification. Methods in enzymology. Published by Deutscher M., Academic Press, 1990”. The biological material is separated or eluted from the solid support containing the peptide or peptides, pepβ2GPI, using a buffer having a pH between 2 and 11.5, having an NaCl concentration between 0 and 5M, advantageously with a 0.1 mol/liter glycine-HCl buffer having a pH of 2.5.

The detection and/or identification and/or quantification of the infectious compounds attached to the peptide (s), pepβ2GPI, can be done by any known means such as those using detection and/or identification and/or quantification by antibodies, described in “Current Protocols in Immunology by Editions Coligan J., Bierer B., Margulies D., Shevach E., Strober W., and Coico R., Wiley Interscience, 2013”. The term “antibody” refers to polyclonal or monoclonal antibodies. The term “monoclonal antibody” refers to an antibody composition consisting of a homogeneous population of antibodies. This term is not limited with regard to the species producing this antibody, the source of its origin, or the manner in which it was produced. The detection and/or identification and/or quantification of bacteria attached to the peptide (s), pepβ2GPI, is preferably carried out using an antibody specifically recognizing antigens, preferably of a lipid nature. or protein, of infectious compounds. In known manner, this antibody can be conjugated to an enzymatic marker, colloidal gold, to a radioactive, fluorescent or luminescent tracer. Excess antibody is removed by washing. Then added, in a known manner, in the case where the antibody is coupled to an enzymatic marker, a specific substrate for the enzyme conjugated to the antibody, substrate which transforms, under fixed conditions, into a colored product. The formation of said colored compound indicates the presence of the desired infectious compound and allows its identification as well as its quantification.

The detection and/or identification and/or quantification of the infectious compounds attached to the peptide (s), pepβ2GPI, can be done by any known means such as those using detection and/or identification and/or quantification by culture and staining methods.

The detection and/or identification and/or quantification of the infectious compounds attached to the peptide (s), pepβ2GPI, can be done by any known means such as those using detection and/or identification and/or quantification by methods based on nucleic acid technology, such as sequencing and/or detection and/or identification and/or quantification of specific nucleic acids by hybridization with a labeled probe or by a chain reaction obtained with a polymerase (technique called “PCR” or “polymerase chain reaction”) described in “Current Protocols in Molecular Biology. Edited by Ausubel F., Brent R., Kingston R., Moore D., Seidman J., Smith J. & Struhl K., Wiley Interscience, 2003”.

The detection and/or identification and/or quantification of the infectious compounds attached to the peptide (s), pepβ2GPI, can be done by any known means such as those using detection and/or identification and/or quantification by ATPmetry.

The examples given below, purely by way of non-limiting illustration of the invention will make it possible to better understand the invention.

### EXAMPLE 1: BINDING OF A VIRUS ON MAGNETIC MICROBEADS COATED WITH PEPβ2GPI

The virus used is the ISAV virus (Infectious salmon anemia virus), responsible for anemia in salmon, originating from a culture supernatant.

The microbeads, intended to fix the virus, which are used are magnetic microbeads sold by the company MERCK under the name “Estapor® super-paramagnetic microspheres” which have a diameter between 0.300 and 0.500 nm.

The pepβ2GPI were grafted onto the microbeads according to the supplier's recommendations. In summary, the microbeads are suspended in phosphate buffer at a pH of 6.0. The beads are then activated, for 15 minutes, in the presence of 1-ethyl-3-(3-Dimethylaminopropyl) carbodiimide and N-hydroxysuccinimide. After washing with HCl, 2 mM, the beads are suspended in a phosphate buffer at a pH of 7.5, containing the pepβ2GPI. The concentration of pepβ2GPI in this coupling buffer is 20 mg/L; the microbeads are incubated in the buffer with gentle and constant shaking at a temperature of 25° C. for 3 hours. The microbeads are then centrifuged at 1,500 rpm and the supernatant is removed; the centrifugation pellet is suspended in the same buffer as that used for the coupling of pepβ2GPI, which forms the suspension of microbeads coated with pepβ2GPI which we want to test.

One hundred microliters of viral culture supernatant to be studied are placed in an Eppendorf tube with 900 microliters of Tris buffer at a pH of 7.5 and 10 microliters of microbeads. The tubes are shaken horizontally to mix the microbeads well, and each tube is incubated at 37° C. or at room temperature (RT=22° C.) for 30 minutes. In each tube, the microbeads are then separated from the liquid phase by means of a magnet, placed externally against the wall of the tube, and the viral RNA is extracted. The beads are brought into contact with a viral lysis buffer, included in an RNA extraction kit: QIAamp RNA viral kit, sold by QIAGEN and the viral RNA is isolated according to the seller's recommendations.

The RNA is then transcribed into complementary DNA, in the presence of reverse transcriptase, according to the following protocol:

| | name or type | trademark or reference | | Mix for 10 μL RT |
|---|---|---|---|---|
| primers | N6 100 μM 0.2 μg/μl | FermentasThermo S0142 | primers | 1.0 |
| dNTP | dNTP 10 mM | Euromedex | Mix | 1.0 |
| buffer 5X | First Strand buffer | Invitrogen ref 28025-021 | RT Mix | 4.0 |
| DTT | DTT | Invitrogen ref 28025-021 | | 2.0 |
| enzyme | M-MLV RT | Invitrogen ref 28025-021 | | 0.5 |
| RNAsin | RNase OUT | Invitrogen ref 10777-019 | | 0.2 |
| Water | Water PCR | Hyclone SH30538.02 | | 1.3 |

| | per tube | |
|---|---|---|
| primers Mix | 2.00 | μL |
| sample/controls | 10.00 | μL |
| heating | 65 | ° C. |
| time | 5 | min |
| RT mix | 8.00 | μL |
| program | RT | |
| | 10 minutes á 25° C. | |
| | 50 minutes á 37° .C. | |
| | 15 min á 70° C., hold 4-20° C. | |
| Pure cDNA (after RT) | 4 | μL |
| addition of water for diluted cDNA | 36 | μL |

Finally, the complementary DNA (cDNA) is amplified by chain reaction using a polymerase and quantified according to the following protocol:

| | name or type | trademark or reference | Mix by PCR |
|---|---|---|---|
| water | water PCR | Hyclone SH30538.02 | 0.60 |
| primer 1 | ISAV_F 10 μM | Eurogentec forward | 0.20 |
| primer 2 | ISAV_R 10 μM | Eurogentec reverse | 0.20 |
| mix 2X | 480 | Roche ref. 04707516001 | 5.00 |

| | | |
|---|---|---|
| PCR mix | 6.0 | μL |
| samples or | 4.0 | μL |
| controls | | |
| Hybridization | 60 | ° C. |
| temperature | | |
| Fluorescence | 483-533 | nm |

| | |
|---|---|
| program | SYBRgreen1 384 |

| | |
|---|---|
| 95° C. - 5 minutes | |
| 95° C. - 10 seconds | 45 cycles |
| Th ° C. - 10 seconds | |
| 72° C. - 10 seconds | |
| 95° C. - 5 seconds ; 65° C. - 1 minute | |
| 65° C. à 97° C. (0.11° C./s) ; 40° C. - 30 seconds | |

The primers used are:

primers ISAV-F (sense)
3′-CTACACAGCAGGATGCAGATGT-5′
and,
ISAV-R primers (antisense)
3′-CAGGATGCCGGAAGTCGAT-5′

The results obtained are summarized in Table 1 below. They show that all of the tested peptides are effective in capturing the ISAV virus.

TABLE 1
Capture of the ISAV virus by the peptides pepβ2GPI

| Beads | ISAV copies | % compared to β2GPI |
|---|---|---|
| β2GPI | 55 800 | |
| P4 | 61 150 | (110%) |
| P1 | 72 300 | (130%) |
| P2 | 62 700 | (112%) |
| P3 | 65 550 | (117%) |
| P8 | 63 100 | (113%) |
| P9 | 63 800 | (114%) |
| P10 | 58 400 | (105%) |
| P11 | 78 350 | (140%) |

### EXAMPLE 2: BINDING OF A VIRUS TO MAGNETIC MICROBEADS COATED WITH PEPβ2GPI

The virus used is the IPN virus (Infectious pancreatic necrosis virus), responsible for necrosis of the pancreas in salmon, originating from a culture supernatant. The same protocol as described in example 1 was carried out.

The primers used are:

Primers IPNV-F (sense)
5′-TCTCCCGGGCAGTTCAAGT-3′
Primers IPNV-R (antisense)
5′-CGGTTTCACGATGGGTTGTT-3′

The results obtained are summarized in Table 2 below. They show that all of the tested peptides are effective in capturing the IPN virus.

TABLE 2
Capture of the IPN virus by the peptides pepβ2GPI

| Beads | IPN Copies | % compared to β2GPI |
|---|---|---|
| β2GPI | 12 | — |
| P4 | 12 | (100%) |
| P1 | 14 | (117%) |
| P2 | 6 | (50%) |
| P8 | 32 | (267%) |
| P10 | 8 | (67%) |
| P11 | 21 | (175%) |

### EXAMPLE 3: BINDING OF A VIRUS TO MAGNETIC MICROBEADS COATED WITH PEPβ2GPI

The virus used is the HCV virus (hepatitis C virus), present in the plasma of patients. The same protocol as described in example 1 was carried out.

The primers used are:

Primers KY80 (sense)
5′-GCAGAAAGCGTCTAGCCATGGCGT-3′
Primers KY78 (antisense)
5′-CTCGCAAGCACCC TATCAGGCAGT-3′

The results obtained are summarized in Table 3 below. They show that all of the tested peptides are effective in capturing the HCV virus.

TABLE 3
Capture of the HCV virus by the peptides pepβ2GPI

| Beads | HCV/run copies | % compared to β2GPI |
|---|---|---|
| β2GPI | 113 | — |
| P4 | 83 | (73%) |
| P1 | 184 | (163%) |
| P2 | 81 | (72%) |
| P8 | 216 | (191%) |
| P9 | — | — |
| P10 | 98 | (87%) |
| P11 | 97 | (86%) |

### EXAMPLE 4 BINDING OF A VIRUS TO ELISA-TYPE MICROTITER PLATES

The desired compound is an endogenous human retrovirus antigen (HERV: human endogenous retrovirus). His research was carried out on serum from patients with autoimmune pathologies as well as on serum from healthy donors.

The support used is a microtiter plate of the ELISA type, with 96 wells and flat bottom, sold by the company “DYNATECH”. Four samples of serum from healthy donors and four samples from patients with autoimmune disease were used.

The serum sample is diluted ten times in 50 mM Tris-HCl buffer, pH 7.6±0.05.

100 μl of this solution are placed at the bottom of each well of the microplate.

This is then incubated at +37° C. for 90 minutes. The liquid from each well is then aspirated. Then 300 to 400 μl of phosphate buffer containing 0.01 mole/I of sodium and disodium phosphates and 0.15 mole/I of sodium chloride and having a pH of 7.00±0.05 are introduced into each well.

It is left in contact with the support for 3 minutes and the buffer is aspirated; this washing operation is carried out three times.

Next, 100 μl of a specific murine monoclonal antibody solution directed against the envelope protein of the endogenous HERV-W retrovirus is added per well.

The plate is left at 37° C. for 60 minutes.

Following this incubation, the contents of the wells of the plate are aspirated. 300 to 400 μl of phosphate buffer, described above, are introduced into each well, and after a contact time of 3 minutes, the buffer is aspirated; this washing operation is carried out three times.

Then, 100 μL of a solution of a polyclonal antibody recognizing the murine antibodies are added per well. The plate is left at 37° C. for 60 minutes. Following this incubation, the contents of the wells of the plate are aspirated. 300 to 400 μl of phosphate buffer, described above, are introduced into each well, and after a contact time of 3 minutes, the buffer is aspirated; this washing operation is carried out six times.

100 μl of a solution of o-phenylene diamine, 2HCl in a sodium citrate buffer are added per well. It is left to incubate for 30 minutes at room temperature, then the reaction is stopped by adding to each well 50 μl of 2N H₂ SO₄. The absorbance at 492 nm obtained at the end of the reaction is measured using a plate reader robot.

Results in the following table are expressed in P/2N, P corresponding to the average of the absorbances obtained for a given serum and N corresponding to the average of the absorbances obtained from healthy donors, multiplied by 2.

TABLE 4
Capture of HERV by the peptides pepβ2GPI
P/2N

| | β2GPI | P8 | P11 |
|---|---|---|---|
| 100s | 4.9 | 4.111 | 4.039 |
| 99s | 0.974 | 0.731 | 0.977 |
| 45s | 1.112 | 1.129 | 1.228 |
| 6s | 0.928 | 1.207 | 1.185 |
| DS 45s | 0.69 | 0.561 | 0.77 |
| DS 46s | 0.79 | 0.294 | 0.718 |
| DS 60s | 0.637 | 0.189 | 0.631 |
| DS 61s | 0.498 | 0.594 | 0.727 |

DS: healthy donors

For two of the four sick subjects, and not for the four healthy subjects, the HERV envelope antigen was effectively revealed for the two peptides tested.

### EXAMPLE 5: BINDING OF BACTERIA TO MAGNETIC MICROBEADS COATED WITH PEPβ2GPI

The bacteria used is a strain of Escherichia coli (E. coli), B6094, from a clinical isolate. A preculture is incubated at 37° C. for 16 h in LB medium (Luria Bertani) having the following composition:

| Bacto tryptone | 10 | g |
|---|---|---|
| Yeast extract | 5 | g |
| NaCI | 10 | g |
| pH | 7.5 | |
| Water | qs 1000 | g |

This preculture is used immediately or stored at 4.5° C.

The microbeads intended to bind the bacteria which are used in this example are magnetic microbeads sold by the company MERCK under the name “Estapor® superparamagnetic microspheres” which have a diameter of between 0.300 and 0.500 μm.

These microbeads are suspended in an acetate buffer at a pH of 6.0 containing β2GPI. The concentration of β2GPI in this binding buffer is 100 μg/ml; the microbeads are incubated in the buffer with gentle and constant shaking at a temperature of 25° C. for 3 hours. The microbeads are then centrifuged at 1500 rpm and the supernatant is removed; the centrifugation pellet is suspended in the same buffer as that used for the coupling of β2GPI, which forms the suspension of microbeads coated with β2GPI that has to be tested.

One ml of the culture of bacteria to be studied is placed in a hemolysis tube. 100 μl of buffer are added, the trade name of which is TTGB, then 20 μl of microbeads. The tube is stirred horizontally, to mix the microbeads well, and incubated at 37° C. or at room temperature (TA=22° C.) for half an hour. At the end of the incubation, the microbeads are separated from the liquid phase by means of a magnet, placed externally against the wall of the tube. The microbeads are then washed twice with LB medium and then resuspended in 1 ml of BTS culture medium (Tripticase-Soy broth) having the following formulation:

| | |
|---|---|
| Casein peptone | 17.0 g |
| Soy flour peptone | 3.0 g |
| D(+) - glucose | 2.5 g |
| Sodium chloride | 5.0 g |
| Dipotassium phosphate | 2.5 g |
| Water qs | 1000 g |

pH 7.3

This culture medium is brought to a boil and then autoclaved, to make it sterile, before use. 20 μl of the microbead suspension are taken and spread in a Petri dish containing TS agar (Trypticase-Soya agar) having the following formulation:

| | |
|---|---|
| Casein peptone | 15.0 g |
| Soy flour peptone | 5.0 g |
| Sodium chloride | 5.0 g |
| Agar | 15.0 g |

pH 7.3

The Petri dishes are incubated at +37° C. for 24 hours. The bacteria that have grown on the agar are then counted.

The results obtained are summarized in Table 5 below. They show that all of the tested peptides are effective in capturing E. coli B6054, from a clinical isolate.

TABLE 5
Capture of E. coli B6054 by the peptides pepβ2GPI

| Beads | % compared to β2GPI |
|---|---|
| β2GPI | 100 |
| P4 | 44 |
| P1 | 92 |
| P2 | 31 |
| P8 | 73 |
| P10 | 34 |
| P11 | 91 |

### EXAMPLE 6 BINDING OF BACTERIA TO MAGNETIC MICROBEADS COATED WITH PEPβ2GPI

The used bacteria were obtained from a collection strain of Escherichia coli (E. coli), ATCC 8739. The same experiment as that described in the previous example was carried out.

The results obtained are summarized in Table 6 below. They show that all of the tested peptides are effective in capturing E. coli ATCC 8739, from a clinical isolate.

TABLE 6
Capture of E. coli ATCC 8739 by the peptides pepβ2GPI

| Beads | % compared to β2GPI |
|---|---|
| β2GPI | 100 |
| P4 | 18 |
| P1 | 68 |
| P2 | 15 |
| P8 | 65 |
| P10 | 12 |
| P11 | 99 |

### EXAMPLE 7 BINDING OF BACTERIA TO MAGNETIC MICROBEADS COATED WITH PEPβ2GPI

The used bacterium is a strain of Staphylococcus aureus (S. aureus), SA 378804, isolated from the wound of the foot of a diabetic person, Sotto et al., Diabetes Care (2008) 31 (12), p. 2318-24. The same experiment as that described in the previous example was carried out.

The results obtained are summarized in Table 7 below. They show that all of the peptides tested are effective in capturing S. aureus, SA 378804, from a clinical isolate.

TABLE 7
Capture of S. aureus, SA 378804 by the peptides pepβ2GPI

| Beads | % compared to β2GPI |
|---|---|
| β2GPI | 100 |
| P4 | 79 |
| P1 | 115 |
| P2 | 104 |

### EXAMPLE 8 BINDING OF BACTERIA TO MAGNETIC MICROBEADS COATED WITH PEPβ2GPI

The beads, on which β2GPI and peptides derived therefrom were grafted, were used to capture the bacteria present in treated wastewater. This water was recovered at the outlet of the purifier from the Grau-du-Roi treatment plant. The same experiment as that described in the previous example was carried out.

The results obtained are summarized in Table 8 below. They show that all of the tested peptides are effective in capturing bacteria present in treated wastewater from a treatment plant.

TABLE 8
Capture of bacteria present in treated wastewater, by the peptides pepβ2GPI

| Beads | % compared to β2GPI |
|---|---|
| β2GPI | 100 |
| P11 | 105 |

### EXAMPLE 9: BINDING OF BACTERIA TO MAGNETIC MICROBEADS COATED WITH PEPβ2GPI

The beads, on which β2GPI and peptides derived therefrom were grafted, were used for the capture of bacteria present in human blood and highlighted by blood culture.

These blood cultures are carried out by placing a venous blood sample (approximately ml) in aerobic vials of BacT/ALERT®3D type. These flasks are then incubated in an automated system at 35° C. for at least 5 days. The bottles are equipped with a colorimetric detection system with a sensor located at the base of each bottle. Carbon dioxide produced by growing bacteria changes the color of the sensor; this change in color is detected by an automated system and indicates the presence of bacterial growth: these blood cultures are said to be positive. In the BacT/ALERT®3D vials there are particles of activated carbon, which inhibit antibiotics potentially present in the blood of patients, thereby improving the detection of microorganisms. To confirm the presence of bacteria in blood cultures which have been found positive, the hospital performs a culture on blood agar. All the results from hospitals thus make it possible to identify:
positive blood cultures (positive in the automated system and positive in culture),
negative blood cultures (negative in the automated system) and
false positive blood cultures (positive in the automated system and negative in culture).

To test the interaction of microbeads coated with β2GPI and the peptides derived from them, with bacteria present in the blood, 1 ml of blood culture is taken from each sample and placed in a 15 ml tube. 20 μl of microbeads are added and each tube is incubated at 37° C. with horizontal shaking. The tubes are then placed in a magnetic field which retains the microbeads on the wall and the supernatant is removed. The microbeads are then washed twice with sterile PBS, of the same composition as previously indicated; the microbeads are then resuspended in 150 μl of BTS culture medium. Two methods for detecting bacteria captured by microbeads were used: culture on TS agar and PCR For the detection of the bacterial capture by culture, 50 μl of the suspension of microbeads thus obtained are taken and they are spread out in a Petri dish containing TS agar. Petri dishes are incubated in an oven at 37° C. for 24 hours. The bacteria that have grown on the agar were then counted.

The results obtained are summarized in Table 9 below. They show that the tested peptides are effective in capturing the bacteria present in the 4 blood cultures tested.

TABLE 9
Capture of bacteria present in blood cultures, by the peptides pepβ2GPI

| | % compared to β2GPI | | | |
|---|---|---|---|---|
| Beads | Yeast | SCN | E. faecium | S. maltophilia |
| β2GPI | 100 | 100 | 100 | 100 |
| P11 | 89 | 101 | 88 | 29 |

To demonstrate the bacterial capture by PCR, 50 μl of the suspension of microbeads thus obtained are taken and the bacterial DNA is extracted.

The bacteria are lysed by adding 100 μL of “Chelex 30%” (InstaGene Matrix, BioRad). The mixture is incubated for 10 min at 95° C., then centrifuged for 10 min at 10,000 rpm. The DNA-containing supernatant is stored at −20° C.

To 3 μl of extracted DNA are added 47 μl of the amplification solution (Aquapure Genomic DNA Isolation Kit); the final concentrations are as follows:
5 μl of dXTP at 200 mM,
10 μl BUFFER 5×,
5 μl of 2 mM MgCl2,
1 μl of each primer diluted to 10 μM:

27F:
5′-GTGCTGCAGAGAGTTTGATCCTGGCTCAG-3′
1492R:
5′-CACGGATCCTACGGGTACCTTGTTACGACTT-3′

1 μl of Taq polymerase (Fast Start Hight Fidelity, Roche) at 5 u/μl
water for injections qs 50 μl

After homogenization, the reaction mixtures are placed in a thermocycler (Master cycler personal Eppendorf) and subjected to the following program:
An initial denaturation at 95° C. of 2 min
Followed by 30 cycles including:
1 min at 95° C.
30 sec at 62° C.
1 min at 72° C.
Then a final extension of 7 min at 72° C.

Verification of the presence of an amplification product of approximately 1400 bp is done by migration of part of the sample on 1.5% agarose gel in 0.5×TBE buffer containing ethidium bromide at 1 μg/mL. The gel is then visualized under UV light. The PCR products are directly sequenced by automatic sequencing by the company Cogenics (Meylan, France) with the 27F primer.

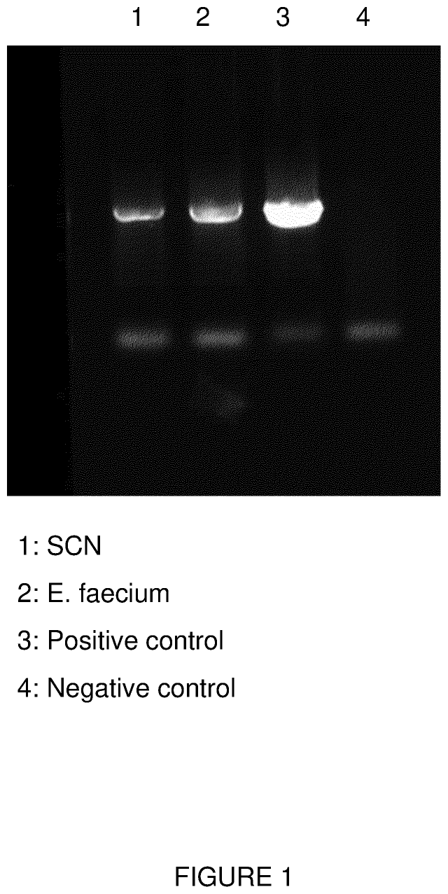

The results obtained are shown in the following FIG. 1. They show that the tested peptides are effective in capturing the bacteria present in the 2 blood cultures tested.

FIG. 1: Capture of bacteria, present in blood cultures by the peptide P11

### EXAMPLE 10: BINDING OF THE HSV AND BVDV VIRUSES ON MAGNETIC MICROBEADS COATED WITH PEPβ2GPI

The viruses tested are the HSV virus (Herpes Simplex virus) and the BVDV virus (Bovine viral Diarrhea Virus).

The same protocol as that described in example 1 was carried out.

The primers used are:

For HSV
HSV-1 (sense)
TGGGACACATGCCTTCTTGG
HSV-1 (antisense)
ACCCTTAGTCAGACTCTGTTACTTACCC
For BVDV
BVDV (sense)
GCCATGCCCTTAGTAGGACTAGC
BVDV (antisense)
CAACTCCATGTGCCATGTACA

The results obtained are summarized in Table 10 below. They show that all the tested peptides are effective in capturing the HSV and BVDV viruses.

TABLE 10
Capture of HSV and BVDV viruses by the pepβ2GPI peptides.

| Peptide | HSV | BVDV |
|---|---|---|
| P4 | 357% | 73% |
| P1 | | 7% |
| P2 | 97% | 181% |
| P3 | | 15% |
| P8 | 112% | 212% |
| P9 | 380% | 85% |
| P10 | 119% | 208% |
| P11 | 123% | 266% |
| P12 | 211% | 125% |
| P13 | 196% | 132% |
| P14 | 353% | 99% |
| P15 | 193% | 91% |
| P16 | 130% | 65% |
| P17 | 137% | 40% |
| P18 | 145% | 78% |
| P19 | 362% | 125% |
| P20 | 229% | 192% |
| P21 | 190% | 116% |
| P22 | 566% | 162% |
| P23 | 169% | 133% |
| P24 | 134% | 64% |
| P25 | 357% | 157% |
| P26 | 137% | 61% |
| P27 | 169% | 164% |
| P28 | 321% | 173% |
| P29 | 180% | 212% |
| P30 | 132% | 226% |
| P31 | 271% | 126% |
| P32 | 176% | 213% |
| P33 | 289% | 169% |
| P34 | 150% | 66% |
| P35 | 148% | 80% |
| P36 | 237% | 146% |
| P37 | 189% | 71% |
| P38 | 102% | 110% |
| P39 | 99% | 76% |

### EXAMPLE 11: BINDING OF BACTERIA AND YEASTS TO MAGNETIC MICROBEADS COATED WITH PEPβ2GPI

The tested bacteria are: C3 E. coli ATCC 11105, C5 S. aureus ATCC 6538, H61 E. coli B6054, C1: E. coli ATCC 8739, H46: C. albicans 1 and H: 60 SCN

The same protocol and primers as those described in example 9 were carried out.

The results obtained are summarized in Table 11 below. They show that all the tested peptides are effective for the bacteria and yeasts.

TABLE 11
Capture of bacteria by the peptides pepβ2GPI.

| | C3 | C5 | | C1: | H46: | |
|---|---|---|---|---|---|---|
| | E. coli | S. aureus | H61 | E. coli | C. | H: |
| | ATCC | ATCC | E. coli | ATCC | albicans | 60 |
| Peptide | 11105 | 6538 | B6054 | 8739 | 1 | SCN |
| P4 | 26% | 73% | 85% | | | |
| P1 | 30% | 56% | 71% | | | |
| P2 | 30% | 80% | 104% | | | |
| P3 | 42% | 65% | 88% | | | |
| P8 | 38% | 72% | 90% | | | |
| P9 | 30% | 82% | 85% | 8% | 100% | 67% |
| P10 | 45% | 66% | 79% | 6.17% | 97.9% | 49.0% |
| P11 | 43% | 63% | 90% | | | |
| P12 | 54% | 60% | 72% | 8.03% | 97.3% | 51.7% |
| P13 | 56% | 72% | 92% | 6.59% | 98.7% | 49.3% |
| P14 | 51% | 24% | 73% | 8.52% | 118.9% | 38.4% |
| P15 | 55% | 117% | 79% | 7.94% | 88.2% | 56.9% |
| P16 | 75% | 84% | 110% | 8.66% | 80.4% | 53.1% |
| P17 | 66% | 134% | 83% | 6.97% | 66.4% | 58.0% |
| P18 | 61% | 94% | 82% | 5.35% | 101.8% | 47.1% |
| P19 | 63% | 91% | 75% | 10.06% | 56.5% | 41.7% |
| P20 | 80% | 83% | 74% | 10.66% | 78.2% | 53.3% |
| P21 | 80% | 86% | 65% | 10.12% | 41.6% | 52.3% |
| P22 | 62% | 94% | 78% | 11.75% | 102.5% | 61.1% |
| P23 | 59% | 90% | 65% | 10.69% | 58.7% | 46.4% |
| P24 | 65% | 67% | 94% | 7.97% | 61.8% | 36.6% |
| P25 | 60% | 88% | 80% | 11.58% | 48.3% | 34.3% |
| P26 | 81% | 72% | 97% | 9.47% | 102.9% | 36.6% |
| P27 | 81% | 106% | 99% | 14.07% | 72.2% | 45.6% |
| P28 | 30% | 95% | 72% | 7.84% | 58.7% | 25.0% |
| P29 | 24% | 107% | 85% | 13.08% | 50.4% | 42.7% |
| P30 | 30% | 95% | 75% | 6.61% | 58.7% | 18.0% |
| P31 | 29% | 81% | 84% | 10.70% | 53.6% | 32.6% |
| P32 | 39% | 94% | 75% | 8.53% | 39.2% | 39.3% |
| P33 | 39% | 98% | 94% | 6.04% | 73.1% | 32.8% |
| P34 | 49% | 97% | 83% | 16.80% | 75.9% | 66.3% |
| P35 | 52% | 97% | 85% | 11.95% | 62.1% | 68.8% |
| P36 | 45% | 102% | 64% | 17.09% | 24.4% | 98.4% |
| P37 | 57% | 83% | 91% | 11.37% | 23.1% | 66.5% |
| P38 | 70% | 94% | 83% | 4.40% | 62.1% | 54.3% |
| P39 | | | 87% | 9.1% | 45.7% | 77.8% |

1. A peptide chosen from peptides capable of binding microorganisms chosen from
P1: SSLAFWK
P2: CKNKEKKC in cyclic form (presence of a disulfide bridge)
P3: CKNKEKKC in linear form (absence of disulfide bridge)
alone or linked to each other to give the following molecules
P4: CKNKEKKCGGSSLAFWK; or
P5: R—X—R′
P6: H—(R)₂—K or
P7: H—[(R)₂—K)₂—K
Wherein
R and R′, identical or different, can be P1: SSLAFWK; or P2: CKNKEKKC in cyclic form; or P3: CKNKEKKC in linear form; and
X can be one spacer chosen from
Sp1: —CO—NH—(CH₂)m-NH—CO—(CH₂)n-CO—NH—(CH₂)p-CO—NH—; wherein m can be an equal integer which can have any one of the values 1, 2, 3, 4, 5, 6, 7, 8, 9 or 10, preferably 4, 5, 6, 7 or 8, more preferably 6; n can be an equal integer which can have any one of the values 1, 2, 3, 4 or 5, preferably 2, 3 or 4, more preferably 3; and p can be an equal integer which can have any one of the values 1, 2, 3, 4, 5, 6, 7, 8, 9 or 10, preferably 4, 5, 6, 7 or 8, more preferably 5;
Sp2: —CO—(CH₂)m-NH—CO—(CH₂)n-CO—NH—(CH₂)p-NH—; wherein m and p can be an equal integer which can have independently any one of the values 1, 2, 3, 4, 5, 6, 7, 8, 9 or 10, preferably 4, 5, 6, 7 or 8, more preferably 5; and n can be an equal integer which can have any one of the values 1, 2, 3, 4 or 5, preferably 2, 3 or 4, more preferably 3;
Sp3: —CO—(CH₂)m-O—(CH₂)n-NH—CO—(CH₂)p-O—(CH₂)q-CO—NH—(CH₂)r-O—(CH₂)s-NH—; wherein m, and q can be an equal integer which can have independently any one of the values 1, 2 or 3, preferably 2; and p and q can be an equal integer which can have independently any one of the values 1 or 2, preferably 1;
Sp4: —CO—(CH₂)m-NH—CO—(CH₂)n-CZ—(CH₂)p-NH—CO—(CH₂)q-NH—, wherein Z=cyclic C₅H₁₀ and wherein m and q can be an equal integer which can have independently any one of the values 1, 2, 3, 4, 5, 6, 7, 8, 9 or 10, preferably 4, 5, 6, 7 or 8, more preferably 5; and n and p can be an equal integer which can have independently any one of the values 1 or 2, preferably 1;
Sp5: —CO—(CH₂)m-NH—CO—NH—(CH₂)n-CO—NH—(CH₂)p-NH—; wherein m and p can be an equal integer which can have independently any one of the values 1, 2, 3, 4, 5, 6, 7, 8, 9 or 10, preferably 4, 5, 6, 7 or 8, more preferably 6; and n can be an equal integer which can have any one of the values 2, 3, 4, 5 or 6, preferably 3, 4, 5, more preferably 4;
Sp6: —CO—(CH₂)m-NH—CO—(CH₂)n-NH—CO—(CH₂)p-NH—; wherein m, n and p can be an equal integer which can have independently any one of the values 1, 2, 3, 4, 5, 6, 7, 8, 9 or 10, preferably 3, 4, 5, 6 or 7, more preferably 5;
Sp7: —CO—(CH₂)m-NH—CO—(CH₂)n-NH—; wherein m and n can be an equal integer which can have independently any one of the values 1, 2, 3, 4, 5, 6, 7, 8, 9 or 10, preferably 3, 4, 5, 6 or 7, more preferably 5;
Sp8: —CO—(CH₂)m-CO—NH—(CH₂)n-NH—CO—(CH₂)p-NH—; wherein m can be an equal integer which can have any one of the values 1, 2, 3, 4, 5, 6, 7, 8, 9 or preferably 4, 5, 6, 7 or 8, more preferably 5; n can be an equal integer which can have any one of the values 1, 2, 3, 4 or 5, preferably 2, 3 or 4, more preferably 3; and p can be an equal integer which can have any one of the values 1, 2, 3, 4, 5, 6, 7, 8, 9 or 10, preferably 4, 5, 6, 7 or 8, more preferably 6;
Sp9: —CO—(CH₂)m-NH—CO—(CH₂)n-NH—CO—(CH₂)p-NH—; wherein m can be an equal integer which can have any one of the values 1, 2, 3, 4, 5, 6, 7, 8, 9 or preferably 4, 5, 6, 7 or 8, more preferably 5; n can be an equal integer which can have any one of the values 1, 2, 3, 4 or 5, preferably 2, 3 or 4, more preferably 3; and p can be an equal integer which can have any one of the values 1, 2, 3, 4, 5, 6, 7, 8, 9 or 10, preferably 4, 5, 6, 7 or 8, more preferably 6;
Sp10: —CO—(CH₂)m-O—(CH₂)n-O—(CH₂)p-O—(CH₂)q-O—(CH₂)r-O—(CH₂)s-NH—; wherein m, n, p, q, r, and s can be an equal integer which can have independently any one of the values 1, 2 or 3, preferably 2;
SP11: —CO—(CH₂)m-NH—CO—(CH₂)n-NH—CO—(CH₂)p-NH—CO—(CH₂)q-NH—CO—(CH₂)r-NH—CO—(CH₂)s-NH—; wherein m, n, p, q, r, and s can be an equal integer which can have independently any one of the values 1 or 2, preferably 1;
SP12: —CO—CH₂—NH—CO—CHCH₂OH—NH—CO—CH₂—NH—CO—CHCH₂OH—NH—CO—CH₂—NH—CO—CHCH₂OH—NH—;
SP13: —CO—(CH₂)m-NH—CO—C[NH—CO—(CH₂)n-NH-](CH₂)p-NH—CO—(CH₂)q-NH—; wherein m, n, p and q, can be an equal integer which can have independently any one of the values 1, 2, 3, 4, 5, 6, 7, 8, 9 or 10, preferably 4, 5, 6, 7 or 8, more preferably 5;
it being understood that in P1, P2, P3, any amino acid, but only one each time, can be substituted by any other amino acid.

2. The peptide according to claim 1 chosen from
P4: CKNKEKKCGGSSLAFWK
P1: SSLAFWK
P2: CKNKEKKC in cyclic form (presence of a disulfide bridge)
P3: CKNKEKKC in linear form (absence of disulfide bridge)
P8: 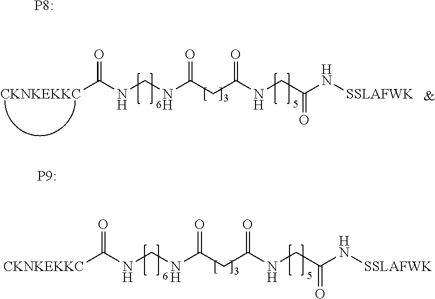
P10: H-(SSLAFWK)2-K—NH₂
P11: H-((SSLAFWK)2-K)2-K—NH₂; or
peptides comprising A: CKNKEKKC and B: SSLAFWK separated by different spacers:

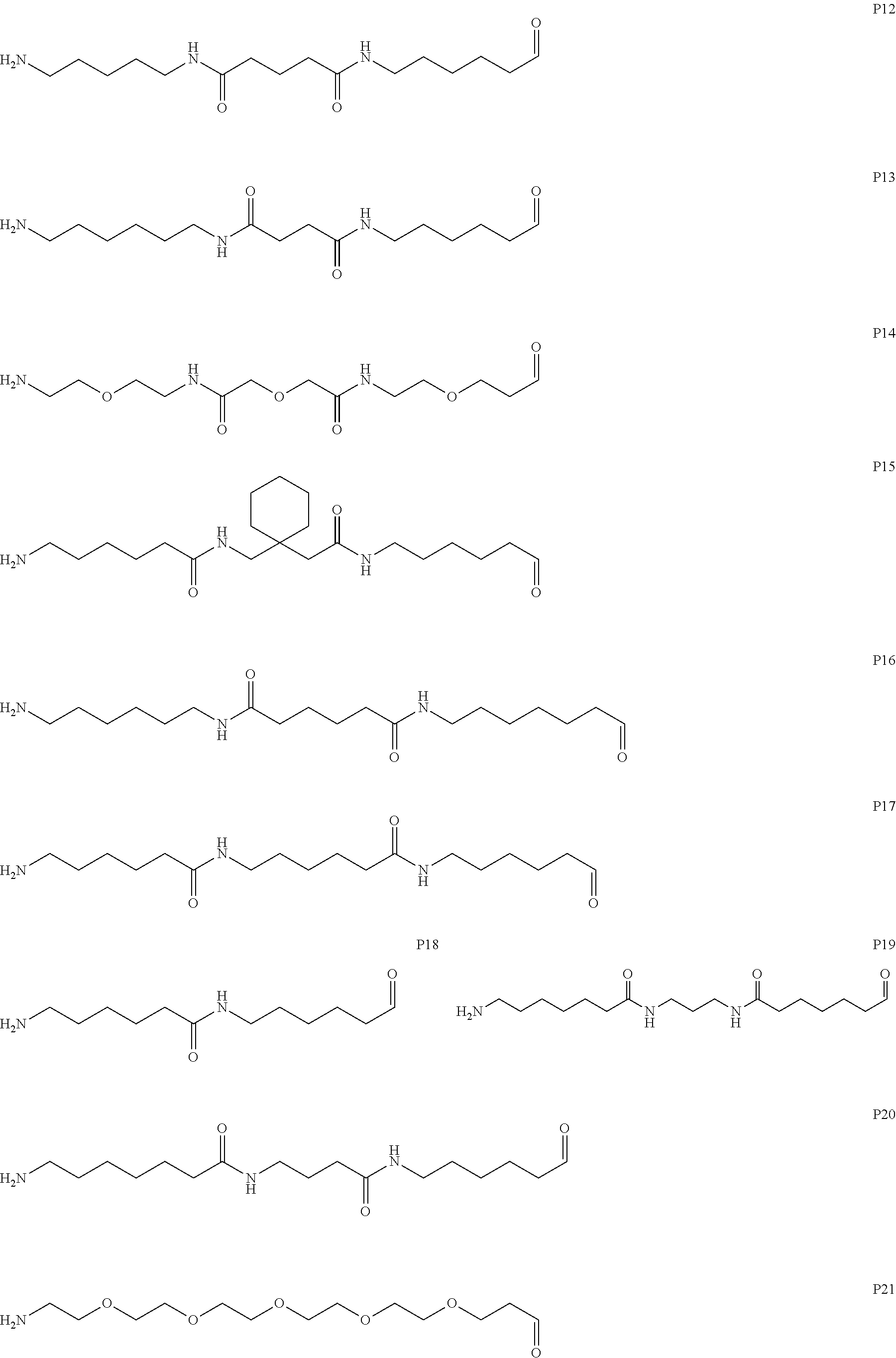

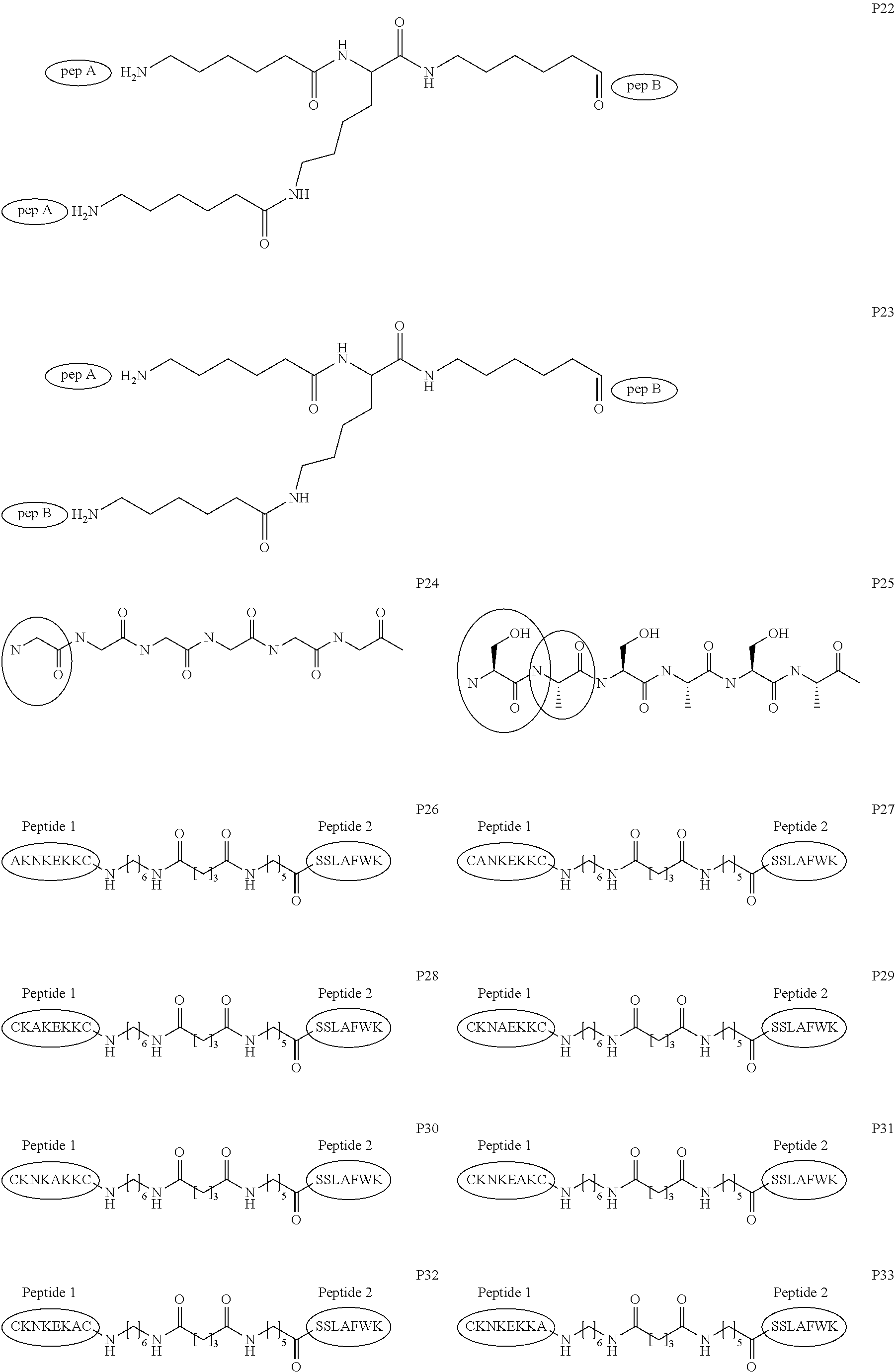

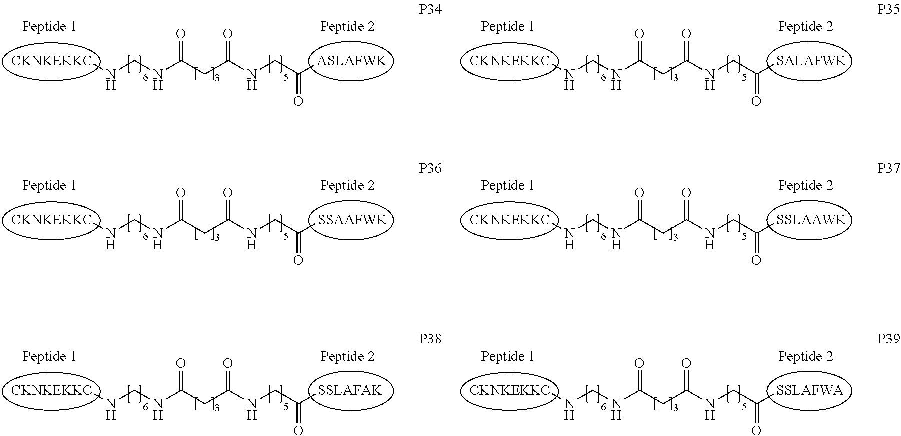

3. The peptide according to claim 1 for its use to capture a microorganism present in biological material.

4. The peptide according to claim 3, characterized in that said biological material microorganism is a bacterium, Gram+ and Gram−, a DNA or RNA virus or a fungus.

5. A microorganism sensor, characterized in that it comprises at least one peptide according to claim 1, coupled to a solid support.

6. An in vitro method for capturing a microorganism present in a biological material characterized in that it comprises
a first step of bringing a microorganism sensor according to claim 5 into contact with biological material potentially containing microorganisms;
a second stage of incubation of the sensor and the biological material for a period of time, between 2 minutes and 24 hours;
a third step of separation of the biological material and the sensor;
a fourth step of detection and/or identification and/or quantification of the microorganisms attached to the sensor.
